SEQUENCE LISTING (13 sequences)

SEQ ID NO: 1 - RNA, 21 nt, Artificial Sequence
  Fully synthetic
agggagggacgggggcugugc

SEQ ID NO: 2 - DNA, 89 bp, Artificial Sequence
  Fully synthetic
gccggcgcccgagctctggctccgtgtcttcactcccgtgcttgtccgaggagggagggagggacgggggctgtgctggggcagctgga

SEQ ID NO: 3 - DNA, 50 bp, Artificial Sequence
  Fully synthetic
gtgcagggtccgaggtcagagcgacgtgcgcaattttttttttttttttt

SEQ ID NO: 4 - DNA, 20 bp, Artificial Sequence
  Fully synthetic
cgcggagggagggacggggg

SEQ ID NO: 5 - DNA, 18 bp, Artificial Sequence
  Fully synthetic
acgtgcagggtccgaggt

SEQ ID NO: 6 - DNA, 19 bp, Artificial Sequence
  Fully synthetic
ctgactccgagaaggcgtc

SEQ ID NO: 7 - DNA, 22 bp, Artificial Sequence
  Fully synthetic
gtattcaccacgtttgtggagc

SEQ ID NO: 8 - DNA, 22 bp, Artificial Sequence
  Fully synthetic
actggacatcacagcagaatga

SEQ ID NO: 9 - DNA, 20 bp, Artificial Sequence
  Fully synthetic
cttcgtgaccagctgtcctt

SEQ ID NO: 10 - DNA, 20 bp, Artificial Sequence
  Fully synthetic
gcagccaggagacaagaact

SEQ ID NO: 11 - DNA, 20 bp, Artificial Sequence
  Fully synthetic
agcgcttcactctttcacga

SEQ ID NO: 12 - DNA, 22 bp, Artificial Sequence
  Fully synthetic
ttgttacaggaagtcccttgcc

SEQ ID NO: 13 - DNA, 22 bp, Artificial Sequence
  Fully synthetic
atgctatcacctcccctgtgtg

(19) United States
(12) Patent Application Publication
(10) Pub. No.: US 2019/0153446 A1
(43) Pub. Date: May 23, 2019

(54) MIR-149-3P AND METHOD FOR TREATING METABOLIC DISEASE USING THE SAME

(71) Applicant: Henan University, Kaifeng (CN)

(72) Inventors: Weidong CHEN, Kaifeng (CN); Xiaobo NIE, Kaifeng (CN); Yandong WANG, Beijing (CN); Yun ZHOU, Kaifeng (CN); Xinrui LV, Kaifeng (CN)

(21) Appl. No.: 16/264,585

(22) Filed: Jan. 31, 2019

Related U.S. Application Data
(63) Continuation-in-part of application No. PCT/CN2017/081994, filed on Apr. 26, 2017.

(30) Foreign Application Priority Data
Aug. 11, 2016 (CN) 201610655996.9

(51) Int. Cl. C12N 15/113; C12Q 1/6883

(52) U.S. Cl. CPC C12N 15/113; C12Q 1/6883; C12N 2310/141; C12Q 2600/178; C12Q 2600/158

(57) ABSTRACT

MicroRNA, including one of or a combination of the following components: (a) a pri-miRNA of miR-149-3p; (b) a pre-miRNA of miR-149-3p; (c) a mature miRNA of miR-149-3p; (d) a miR-149-3p derivative; (e) a 18-26 nucleotides miRNA having a sequence of 5′-AGGGAGG-3′; and (f) a derivative of the 18-26 nucleotides miRNA of (e). Also provided is a method for treating a metabolic disease. The method includes employing a DNA sequence encoding miR-149-3p as a target gene, constructing an overexpression vector of the miR-149-3p, preparing a pharmaceutical composition including the overexpression vector of the miR-149-3p, and administering the pharmaceutical composition to a patient in need thereof.

### CROSS-REFERENCE TO RELAYED APPLICATIONS

This application is a continuation-in-part of International Patent Application No. PCT/CN2017/081994 with an international filing date of Apr. 26, 2017, designating the United States, now pending, and further claims foreign priority benefits to Chinese Patent Application No. 201610655996.9 filed Aug. 11, 2016. The contents of all of the aforementioned applications, including any intervening amendments thereto, are incorporated herein by reference. Inquiries from the public to applicants or assignees concerning this document or the related applications should be directed to: Matthias Scholl P C., Attn.: Dr. Matthias Scholl Esq., 245 First Street, 18th Floor, Cambridge, Mass. 02142.

### BACKGROUND

This disclosure relates to microRNA and a method for treating a metabolic disease using the same.

MicroRNA (abbreviated miRNA) is a small non-coding RNA molecule containing about 22 nucleotides that functions in RNA silencing and post-transcriptional regulation of gene expression. For example, microRNA-122, microRNA-370 and microRNA-378/378* are post-transcriptional regulators of lipid metabolism; microRNA-33a and microRNA-33b are involved in the regulation of cholesterol and lipid metabolism; microRNA-130a, microRNA-200 and microRNA-410 are involved in the regulation of insulin secretion.

### SUMMARY

The disclosure provides microRNA and a method for treating a metabolic disease using the same.

Provided is microRNA, comprising one of or a combination of the following:
(a) a pri-miRNA of miR-149-3p;
(b) a pre-miRNA of miR-149-3p;
(c) a mature miRNA of miR-149-3p;
(d) a miR-149-3p derivative;
(e) a 18-26 nucleotides miRNA comprising a sequence of 5′-AGGGAGG-3′; and
(f) a derivative of the18-26 nucleotides miRNA of (e).

The derivative in (d) and/or (f) can be a cholesterol modifier, a locked nucleic acid modifier, a nucleotide modifier, a glycosylation modifier, a hydrocarbon modifier, a nucleic acid modifier, or a combination thereof.

In (e), the sequence of 5′-AGGGAGG-3′ can be located in positions 2-8 of the miRNA; and the 18-26 nucleotides miRNA can comprise more than 50% of activities of the miR-149-3p.

The mature miRNA of miR-149-3p can comprise a RNA sequence represented by SEQ ID NO: 1, or a derivative thereof, and a DNA sequence encoding the mature miRNA can be represented by SEQ ID NO: 2, or a derivative thereof.

Also provided is a method for treating a metabolic disease, the method comprising employing a DNA sequence encoding miR-149-3p as a target gene, constructing an overexpression vector of the miR-149-3p, preparing a pharmaceutical composition comprising the overexpression vector of the miR-149-3p, and administering the pharmaceutical composition to a patient in need thereof.

The metabolic disease can comprise obesity, fatty liver, hyperlipidemia, hyperuricemia, hypertension, diabetes, atherosclerosis, stroke, or symptoms thereof.

The overexpression vector can comprise a viral expression vector and/or a eukaryotic expression vector; the viral expression vector can comprise an adenovirus vector, an adeno-associated virus vector, a retroviral vector, a herpes virus vector, or a combination thereof; and the eukaryotic expression vector can comprise PCMV-myc expression vector, pcDNA3.0, pcDNA3.1, a modifier thereof, or a combination thereof.

The pharmaceutical composition can be in the form of a granule, a sustained-release agent, a microinjection, a transfectant, a surfactant, or a combination thereof.

The pharmaceutical composition comprising the overexpression vector of the miR-149-3p can be introduced or transfected into the patient's cells or allogeneic cells in vitro, and the cells can be amplified in vitro and then transferred to the patient.

The pharmaceutical composition comprising the overexpression vector of the miR-149-3p can be directly introduced to the patient.

A method of diagnosis of type 2 diabetes, comprising:
1) extracting total microRNAs of claim 1 from a patient's blood and preparing corresponding cDNAs thereof;
2) measuring an expression level of mature microRNAs by fluorescence quantitative PCR; and
3) evaluating the mature microRNAs.

Preparing the corresponding cDNAs can employ a reverse transcription primer as shown in SEQ ID NO: 3.

The fluorescence quantitative PCR can comprise dye detection and/or probe detection.

The fluorescence quantitative PCR can employ a forward primer as shown in SEQ ID NO: 4, and a reverse primer as shown in SEQ ID NO: 5.

Advantages of the microRNA and the use thereof for treating a metabolic disease according to embodiments of the disclosure are summarized as follows. The microRNA can improve the insulin sensitivity, reduce the abnormal accumulation of triglycerides in liver, and reduce the deposition of lipid plaques in blood vessels, thus inhibiting the occurrence and development of metabolic diseases. The microRNA can be used to prepare drugs for the prevention and treatment of metabolic diseases and for the diagnosis and treatment of metabolic diseases. The microRNA can also be used as an auxiliary detection means for the diagnosis of type 2 diabetes.

### BRIEF DESCRIPTION OF THE DRAWINGS

FIG. 1 is fluorescence quantitative PCR results of miRNA mimics-induced overexpression of miR-149-3p in mouse hepatocarcinoma cells as described in the disclosure;

FIG. 2 is fluorescence quantitative PCR results of overexpression of miR-149-3p in mouse hepatocarcinoma cells as described in the disclosure;

FIG. 3 is fluorescence quantitative PCR results of miRNA mimics-induced overexpression of miR-149-3p in obese mice as described in the disclosure;

FIG. 4 is triglyceride levels in the liver measured using a triglyceride test kit;

FIG. 5 is a comparison of staining results of liver and aortic arch in mice; and

FIG. 6 is a comparison of relative expression levels of mature miRNA in type 2 diabetes group and healthy control group; and

FIG. 7 is correlation analysis diagram of relative expression level of mature miRNA and fasting blood glucose level in detected samples.

### DETAILED DESCRIPTION

To further illustrate, embodiments detailing microRNA are described below. It should be noted that the following embodiments are intended to describe and not to limit the disclosure.

Unless otherwise mentioned, the “miR-149-3p” mentioned in the disclosure includes a pri-miRNA of miR-149-3p, a pre-miRNA of miR-149-3p, a mature miRNA of miR-149-3p, or a modifier or derivative thereof.

The term “processing” used in the disclosure refers to the entire biological process of obtaining mature miRNA from DNA. In the eukaryotic cells, the process can be completed automatically and generate a primary miRNA (pri-miRNA), a precursor miRNA (pre-miRNA) and a mature miRNA. The DNA is not limited to the source, and includes but is not limited to chromosome DNA and vector DNA.

The operations in the following examples are normal operations unless otherwise specified.

Materials:

Mouse hepatocarcinoma cell line Hepa1-6: Wuhan BOSTER Biological Technology Co., Ltd.

C57BL/6J mouse: Beijing Vital River Laboratory Animal Technology Co., Ltd.

Tri reagent: Jiangsu Enmo Asai Biotechnology Co., Ltd.

Nuclease-Free Water: Ambion, Inc. U.S.A.

Cholesterol modified miR-149-3p mimics and Cholesterol modified miRNA control: Guangzhou RiboBio Co., Ltd.

Liposome 2000: Invitrogen, Inc., U.S.A.

BioTeke MicroRNA Gene First Chain Synthesis Kit: Beijing Bioteke Corporation

High fat diet (60% kcal Fat): Research Diets, Inc.

Triglyceride (TG enzyme) test kit: Nanjing Jiangcheng Bioengineering Institute

Oil-Red-O Staining Solution: Nanjing Jiangcheng Bioengineering Institute

2×SYBR Green qPCR Mixture: Applied Biosystems

Primer: Sangon Biotech (Shanghai) Co., Ltd.

RNase-free ddH₂O: Ambion, Inc. U.S.A.

miRNA cDNA First Chain Synthesis Kit: Beijing Bioteke Corporation

Unless otherwise specified, the reagents used in the disclosure may be any appropriate commercial reagent; cell lines can be obtained from the market. MiRNA mimics in the following examples are cholesterol modified miRNA with greater stability and longer time-effect in cells.

### EXAMPLE 1: Effect of miR-149-3p on Insulin Signaling Pathway

1. Cell Culture

Mouse hepatocarcinoma cell line Hepa1-6 was cultured in DMEM medium (Thermo, USA). The medium contained 10% fetal bovine serum (Gibco, USA) and penicillin-streptomycin solution (100×). All cells were cultured in a 37° C. incubator with 5% CO₂.

2. Cell Transfection

The Hepa1-6 cells were plated for 20 hours with the density per well about 60%. Thereafter, a miR-149-3p group and a miRNA negative control group were provided for cell transfection. The transfection reagent used was liposome 2000. The transfection method was carried out according to the instructions.

3. Extraction of RNA

After transfection, the cells were cultured for 48 hours and collected. 0.5 mL Tri reagent was added to the cells of each well at room temperature. 5 min later, the bromocresol purple (BCP) solution with 1/10 of the volume of the Tri reagent was added, mixed for 15 seconds and then left at room temperature for 10 min. The mixture was centrifuged under the centrifugal force of 13400 g at 4° C. for 15 min. The supernatant was transferred to a new 1.5 mL centrifugal tube. Isopropanol with equal volume of the supernatant was added. After several times of mixing, the mixture was left alone at room temperature for 10 min, centrifuged under the centrifugal force of 13400 g at 4° C. for 10 min. The supernatant was removed, and 500 μL of 75% ethanol solution (freshly prepared with RNase-free water) was added to clean the RNA. Thereafter, the RNA was centrifuged and precipitated under the centrifugal force of 13400 g at 4° C. for 5 min. The supernatant was removed, and the RNA was dried at room temperature for 5 min. Appropriate nuclease-free water was added to the RNA and the mixture was placed in a 55° C. water bath for 10 min for full dissolution. The absorption values of OD260 and OD280 were determined. It is believed that the A260/A280 of between 1.8 and 2.1 means the total RNA is qualified.

4. Detection of the expression of miR-149-3p and insulin signaling pathway-related genes by fluorescence quantitative PCR

2 μg of RNA was employed as a template. Poly (A) tail was added to miRNA using a miRNA cDNA first strand synthesis kit. The resulting miRNA was reversely transcribed to yield cDNA. The cDNA was employed as a template, and amplified using ABI 7500 fluorescence quantitative PCR instrument in the presence of miR-149-3p primers and PCR 2×SYBR Green qPCR Mixture. The PCR parameters were: 50° C. for 20 seconds; 95° C. for 10 minutes; 95° C. for 1 minute; 60° C. for 1 minute, repeat 40 cycles. The CT values of the amplification of the sample mir-149-3p were measured, and the CT values of the internal reference gene U6 were standardized for correction. Meanwhile, the expression levels of key genes in the insulin signaling pathway, such as protein kinase B2 (Akt2), insulin receptor substrate-1 (Irs1), insulin receptor substrate-2 (Irs2), were detected and corrected with beta-actin as an internal reference gene. The CT values were calculated by 2^{-ΔΔCT} method, and the differences of gene levels in different treatment groups were compared.

The forward primer of the Akt2 is shown in SEQ ID NO: 6; the reverse primer of the Akt2 is shown in SEQ ID NO: 7. The forward primer of the Irs1 is shown in SEQ ID NO: 8; the reverse primer of the Irs1 is shown in SEQ ID NO: 9. The forward primer of the Irs2 is shown in SEQ ID NO: 10; the reverse primer of the Irs2 is shown in SEQ ID NO: 11. The forward primer of the internal reference gene β-actin is shown in SEQ ID NO: 12; the reverse primer of the internal reference gene β-actin is shown in SEQ ID NO: 13.

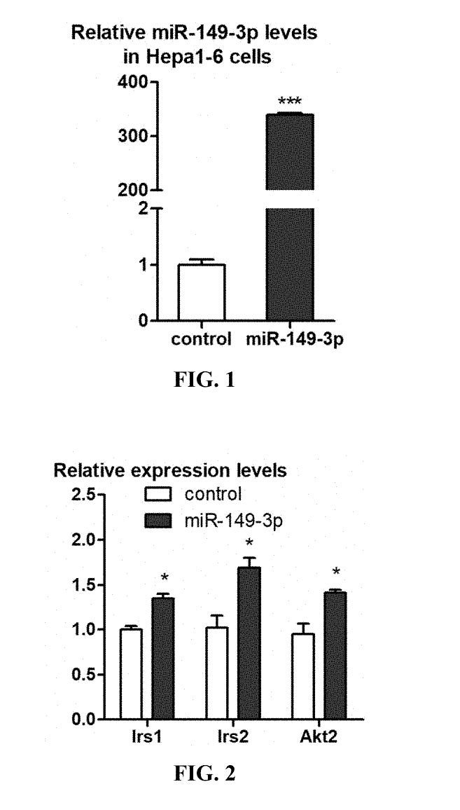

FIG. 1 is fluorescence quantitative PCR results of miRNA mimics-induced overexpression of miR-149-3p in mouse hepatocarcinoma cells.

FIG. 2 shows that after the overexpression of the miR-149-3p in mouse liver cancer cells, the expression of genes related to the insulin signaling pathway is increased and the insulin signaling pathway is activated. β-actin was used as internal reference.

Results: Fluorescence quantitative PCR analysis showed that transfection of 200 pmol of mature miRNA can significantly improve the expression level of miR-149-3p in Hepa1-6 cells, as shown in FIG. 2. Meanwhile, the transcription levels of the key genes comprising Akt2, Irs1 and Irs2 of the insulin signaling pathway are all increased to varying degrees. The experiment showed that increasing the expression level of the miR-149-3p by an exogenous method can activate the insulin signaling pathway and improve the insulin resistance of cells.

### EXAMPLE 2: Effect of Over-Expression of miR-149-3p on Lipid Content in Liver and Vascular Plaque

1. Animal Model Construction

Six-week-old male C57BL/6J mice were fed at 22-24° C. in SPF grade animal room. After 12 hours of circadian rhythm and 12 weeks of feeding with high-fat diet, the mice were injected with miR-149-3p mimics (15 mg/kg) or negative control via the tail vein twice. The mice were fed with high-fat diet for 4 consecutive weeks. The mice were anesthetized with ether and killed. Liver and aortic arch were taken. The use and operation of the mice were conducted in strict accordance with the ethics and animal welfare committee.

2. Detection of the Expression of miR-149-3p In Vivo by Fluorescence Quantitative PCR

The extraction method of the total RNA from tissues was the same as that from cells, except that 1 mL Tri reagent was added to every 100 mg of tissue, and the tissue blocks were crushed on ice. Following the reverse transcription, the changes of the miR-149-3p levels in tissues were detected by fluorescence quantitative PCR.

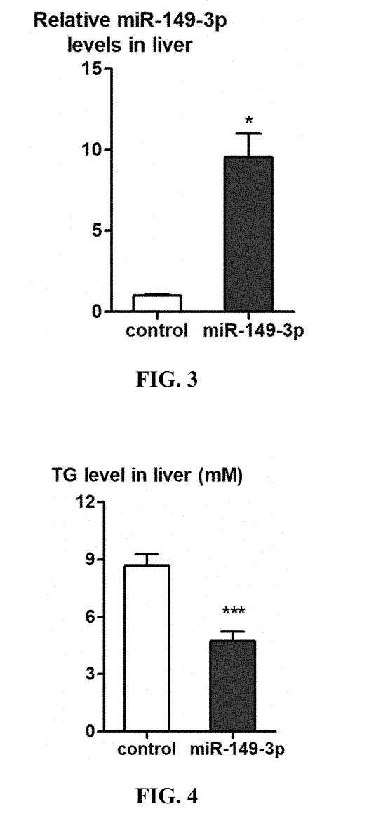

Results: Fluorescence quantitative PCR analysis showed that, as shown in FIG. 3, the expression level of the miR-149-3p molecule in the liver of the mice in the miR-149-3p group (n=4) was significantly increased, nearly 10 times higher than that in the liver of the mice in the control group (n=4). The results showed that injection of exogenous miR-149-3p can ensure the overexpression of the miR-149-3p in tissues.

3. Determination of Fat Content in Mouse Liver

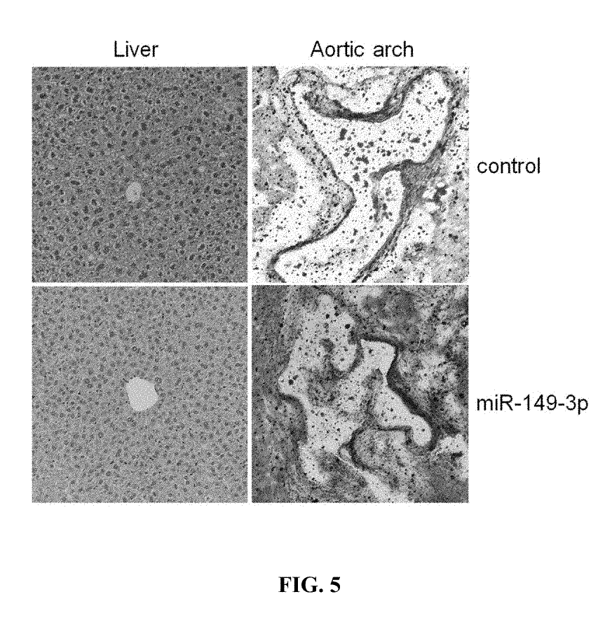

The mouse livers of the two groups were stained with H&E. The liver tissue of the mice fed with high-fat diet showed obvious lipid droplets vacuoles (as shown in FIG. 5), indicating abnormal accumulation of fat in the liver, but the number of lipid droplets vacuoles in the liver decreased significantly after over-expression of the miR-149-3p. Triglyceride levels in the liver were also measured using a triglyceride kit according to the operating instructions. The results showed (as shown in FIG. 4) that overexpression of miR-149-3p significantly reduced triglyceride levels in the liver.

4. Pathological Changes of Aorta in Mice

The mice were anesthetized and killed, and the aortic root was quickly taken for frozen section. The sections were stained with oil red O staining solution. Observe the formation of plaques in different sections, and the images were collected under the microscope. The staining results were shown in FIG. 5. Oil-Red-O stained plaques were observed on the vessel wall of the aortic root of the mice in the negative control group, which was the site of atherosclerosis, while the Oil-Red-O stained plaques in the aortic vessel wall of the mice in the mir-149-3p group were significantly reduced.

Statistical analysis: All data were averaged by three independent repetitive experiments. Standard deviation (SD) was analyzed by GraphPad Prism 5. P<0.05 was considered statistically significant, and *P<0.05; *P<0.01; ***P<0.001.

Insulin signal transduction pathway mainly refers to the activation of the insulin receptor substrate (Irs), the phosphatidylinositol 3 kinase (pi3-k), and the protein kinase B (Akt) after the insulin binds to the receptor on the target cell, thus promoting the storage of substances. Genetic or environmental factors (such as lack of exercise and high-fat diet) can cause abnormal insulin signaling pathway and lead to insulin resistance. Insulin resistance can cause excessive accumulation of triglycerides in the liver, aggravate the degree of insulin resistance, and lead to hyperlipidemia, fatty liver or type 2 diabetes. Insulin resistance can also cause abnormal lipid metabolism and vascular inflammation, and is an independent risk factor for hypertension and atherosclerosis.

The disclosure teaches key miRNAs affecting insulin signaling pathways and lipid metabolism. Experiments have confirmed that overexpression of miR-149-3p in the mouse liver cells can up-regulate the expression of key genes in the insulin signaling pathway and activate insulin signal transduction. Meanwhile, overexpression of miR-149-3p in obese mice induced by high-fat diet can significantly reduce the level of triglycerides in the liver and reduce the abnormal accumulation of lipid droplets in the liver. In addition, the deposition of the lipid plaque in the aortic vessel wall was decreased. The results showed that overexpression of the miR-149-3p in vivo can be a new strategy for the treatment of metabolic diseases, and the miR-149-3p can be a new potential target for the treatment of such diseases.

### EXAMPLE 3: Application of Mature miRNA in the Diagnosis of Type 2 Diabetes

1. Clinical Sample Collection

Since 2015, a large number of peripheral blood samples from patients with type 2 diabetes from Huaihe Hospital affiliated to Henan University and healthy people were collected. The whole process of collection and follow-up experiment conforms to the requirements of medical ethics. Sampling, packing and preservation conditions of the samples are the same. By sorting out the medical records, 30 samples were selected for real-time fluorescence quantitative PCR detection.

Healthy population with fasting blood glucose of between 3.9 and 6.1 mmol/L was defined as a healthy control group.

Population having a fasting blood glucose greater than or equal to 7.0 mmol/L after two consecutive repeated tests was defined as a patient group, which was diagnosed as type 2 diabetes, and the population received no drug treatment.

2. Extraction of Total RNA in Blood

To per 200 μL of fresh blood, 600 μL of Tri reagent was added. The mixture was whirlpool oscillated to lyse the blood cells. 5 min later, the bromocresol purple (BCP) solution with 1/10 of the volume of the Tri reagent was added, mixed for 15 seconds and then left at room temperature for 10 min. The mixture was centrifuged under the centrifugal force of 13400 g at 4° C. for 15 min. The supernatant was transferred to a new 1.5 mL centrifugal tube. Isopropanol with equal volume of the supernatant was added. After several times of mixing, the mixture was left alone at −80° C. for an hour, centrifuged under the centrifugal force of 13400 g at 4° C. for an hour. The supernatant was removed, and 500 μL of 75% ethanol solution (freshly prepared with RNase-free water) was added to clean the RNA. Thereafter, the RNA was centrifuged and precipitated under the centrifugal force of 13400 g at 4° C. for 5 min. The supernatant was removed, and the RNA was dried at room temperature for 5 min. Appropriate nuclease-free water was added to the RNA and the mixture was placed in a 55° C. water bath for 10 min for full dissolution. The absorption values of OD260 and OD280 were determined. It is believed that the A260/A280 of between 1.8 and 2.1 means the total RNA is qualified.

3. Detection of Mature miRNA by Fluorescence Quantitative PCR

2 μg of total RNA was employed as a template. Poly (A) tail was added to miRNA using a miRNA cDNA first strand synthesis kit (BioTeke). A reverse transcription system was prepared after the reaction, as shown in Table 1.

TABLE 1

| | | |
|---|---|---|
| Poly(A) reaction solution | 10 | μL |
| Reverse transcription | 2 | μL |
| primer (10 μM) | | |
| 5 × RT Bufer | 4 | μL |
| dNTP (2.5 mM Each) | 1 | μL |
| RNase inhibitor (40 U/μL) | 1 | μL |
| M-MuLV Reverse | 0.5 | μL |
| transcriptase (200 U/μL) | | |
| RNase-free ddH₂O | 1.5 | μL |
| Total volume | 20 | μL |

The reverse transcription was carried out at 37° C. for 60 minutes to yield cDNA. The cDNA was diluted to 4 ng/μL as a template for quantitative fluorescence PCR. Amplification was carried out on ABI 7500 fluorescent quantitative PCR instrument in the presence of positive primers of mature microRNAs and internal reference gene U6, general reverse primers and 2 *SYBR Green Q PCR Mixture.

The reverse transcription primer of the miRNA as shown in SEQ ID NO: 3; the forward primer is as shown in SEQ ID NO: 4; and the reverse primer is as shown in SEQ ID NO: 5.

The primers for detecting blood miRNA provided in this example are designed based on the poly(A) polymerase tailing method. In certain implementation methods, the real-time fluorescence quantitative PCR primers for detecting the mature miRNA can also be designed according to the stem-loop method. The reaction system was as follows, in which the amplification system of the internal reference gene U6 or mature miRNA was shown in Table 2.

TABLE 2

| | | |
|---|---|---|
| cDNA (20 ng) | 5 | μL |
| Forward primer (10 μM) | 0.5 | μL |
| General reverse primer (10 μM) | 0.5 | μL |
| 2 × SYBR Green qPCR Mixture | 10 | μL |
| RNase-free ddH₂O | 4 | μL |
| Total volume | 20 | μL |

The PCR parameters were: 50° C. for 20 seconds; 95° C. for 10 minutes; 95° C. for 1 minute; 60° C. for 1 minute, repeat 40 cycles. The CT values of the amplification of the sample mature miRNA were measured, and the CT values of the internal reference gene U6 were standardized for correction.

4. Data Processing and Analysis

The ratio of the microRNAs expression in two groups of blood samples was calculated by 2^{-ΔΔCT} method. ΔΔCT=(CT1(miRNA)−CT1(U6))−(CT2(miRNA)−CT2(U6)). CTmiRNA is the CT value of amplification of mature miRNA, CTU6 is the CT value of amplification of the internal reference gene U6, CT1 is the CT value of amplification of the patient group or healthy control group, and CT2 is the CT value of amplification in the healthy control group.

TABLE 3

| Sample | Type 2 | Expression level |
|---|---|---|
| No. | diabetes | of mature miRNA |
| 1 | 0 | 0.97 |
| 2 | 0 | 0.92 |
| 3 | 0 | 1.16 |
| 4 | 0 | 1.82 |
| 5 | 0 | 1.72 |
| 6 | 0 | 0.89 |
| 7 | 0 | 1.26 |
| 8 | 0 | 1.60 |
| 9 | 0 | 1.24 |
| 10 | 0 | 1.13 |
| 11 | 0 | 1.26 |
| 12 | 0 | 1.59 |
| 13 | 0 | 1.43 |
| 14 | 0 | 1.45 |
| 15 | 0 | 0.98 |
| 16 | 1 | 0.77 |
| 17 | 1 | 0.27 |
| 18 | 1 | 0.27 |
| 19 | 1 | 0.73 |
| 20 | 1 | 0.89 |
| 21 | 1 | 0.66 |
| 22 | 1 | 0.51 |
| 23 | 1 | 0.67 |
| 24 | 1 | 0.67 |
| 25 | 1 | 0.68 |
| 26 | 1 | 0.89 |
| 27 | 1 | 0.72 |
| 28 | 1 | 0.83 |
| 29 | 1 | 0.87 |
| 30 | 1 | 0.44 |

In Table 3, “0” represents healthy population, and “1” represents patients with type 2 diabetes.

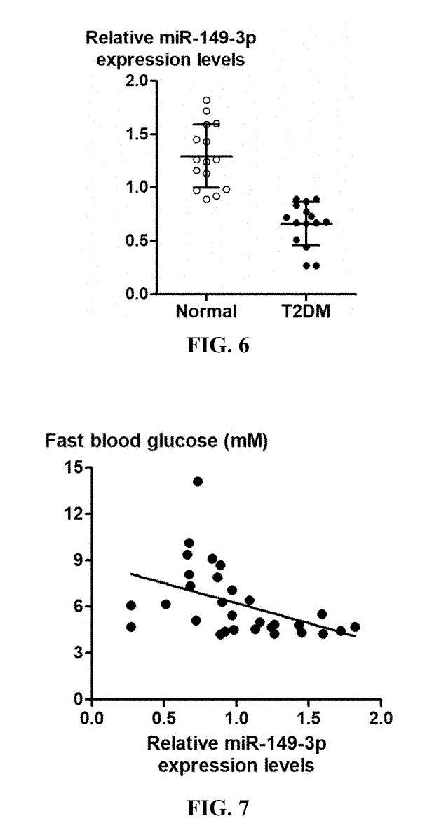

FIG. 6 is a comparison of relative expression levels of mature miRNA in type 2 diabetes group and healthy control group.

FIG. 7 is correlation analysis diagram of relative expression level of mature miRNA and fasting blood glucose level in detected samples.

Statistical analysis by SPSS software showed that the expression levels of mature miRNA in type 2 diabetes patients and healthy control groups were significantly different (P<0.001), and the level of the mature miRNA was significantly negatively correlated with fasting glucose level (R=−0.45, P=0.013), as shown in FIG. 7. P<0.05 was considered statistically significant.

Therefore, it can be concluded that the mature miRNA is significantly decreased in the blood of patients with type 2 diabetes and can be used as a molecular marker for the detection of type 2 diabetes.

The microRNA can improve the insulin sensitivity, reduce the abnormal accumulation of triglycerides in liver, and reduce the deposition of lipid plaques in blood vessels, thus inhibiting the occurrence and development of metabolic diseases. The microRNA can be used to prepare drugs for the prevention and treatment of metabolic diseases and for the diagnosis and treatment of metabolic diseases.

It will be obvious to those skilled in the art that changes and modifications may be made, and therefore, the aim in the appended claims is to cover all such changes and modifications.

What is claimed is:

1. MicroRNA, comprising one of or a combination of the following:
(a) a pri-miRNA of miR-149-3p;
(b) a pre-miRNA of miR-149-3p;
(c) a mature miRNA of miR-149-3p;
(d) a miR-149-3p derivative;
(e) a 18-26 nucleotides miRNA comprising a sequence of 5′-AGGGAGG-3′; and
(f) a derivative of the 18-26 nucleotides miRNA of (e).

2. The microRNA of claim 1, wherein the derivative in (d) and/or (f) is a cholesterol modifier, a locked nucleic acid modifier, a nucleotide modifier, a glycosylation modifier, a hydrocarbon modifier, a nucleic acid modifier, or a combination thereof.

3. The microRNA of claim 1, wherein in (e), the sequence of 5′-AGGGAGG-3′ is located in positions 2-8 of the miRNA; and the 18-26 nucleotides miRNA comprises more than 50% of activities of the miR-149-3p.

4. The microRNA of claim 1, wherein the mature miRNA of miR-149-3p comprises a RNA sequence represented by SEQ ID NO: 1, or a derivative thereof, and a DNA sequence encoding the mature miRNA is represented by SEQ ID NO: 2, or a derivative thereof.

5. A method for treating a metabolic disease, the method comprising employing a DNA sequence encoding miR-149-3p as a target gene, constructing an overexpression vector of the miR-149-3p, preparing a pharmaceutical composition comprising the overexpression vector of the miR-149-3p, and administering the pharmaceutical composition to a patient in need thereof.

6. The method of claim 5, wherein the metabolic disease comprises obesity, fatty liver, hyperlipidemia, hyperuricemia, hypertension, diabetes, atherosclerosis, stroke, or symptoms thereof.

7. The method of claim 5, wherein:
the overexpression vector comprises a viral expression vector and/or a eukaryotic expression vector;
the viral expression vector comprises an adenovirus vector, an adeno-associated virus vector, a retroviral vector, a herpes virus vector, or a combination thereof; and
the eukaryotic expression vector comprises PCMV-myc expression vector, pcDNA3.0, pcDNA3.1, a modifier thereof, or a combination thereof.

8. The method of claim 5, wherein the pharmaceutical composition is in the form of a granule, a sustained-release agent, a microinjection, a transfectant, a surfactant, or a combination thereof.

9. The method of claim 5, wherein the pharmaceutical composition comprising the overexpression vector of the miR-149-3p is introduced or transfected into the patient's cells or allogeneic cells in vitro, and the cells are amplified in vitro and then transferred to the patient.

10. The method of claim 5, wherein the pharmaceutical composition comprising the overexpression vector of the miR-149-3p is directly introduced to the patient.

11. A method of diagnosis of type 2 diabetes, comprising:
1) extracting total microRNAs of claim 1 from a patient's blood and preparing corresponding cDNAs thereof;
2) measuring an expression level of mature microRNAs by fluorescence quantitative PCR; and
3) evaluating the mature microRNAs.

12. The method of claim 11, wherein preparing the corresponding cDNAs employs a reverse transcription primer as shown in SEQ ID NO: 3.

13. The method of claim 11, wherein the fluorescence quantitative PCR comprises dye detection and/or probe detection.

14. The method of claim 11, wherein the fluorescence quantitative PCR employs a forward primer as shown in SEQ ID NO: 4, and a reverse primer as shown in SEQ ID NO: 5.
